(19) United States
(12) Patent Application Publication
(10) Pub. No.: US 2017/0307704 A1
(43) Pub. Date: Oct. 26, 2017

(54) Z-SEGMENTED RF COIL FOR MRI WITH GAP AND RF SCREEN ELEMENT

(71) Applicant: KONINKLIJKE PHILIPS N.V., EINDHOVEN (NL)

(72) Inventors: CHRISTOPH LEUSSLER, EINDHOVEN (NL); CHRISTIAN FINDEKLEE, EINDHOVEN (NL)

(21) Appl. No.: 15/517,287

(22) PCT Filed: Oct. 19, 2015
(86) PCT No.: PCT/EP2015/074084

§ 371 Date: Apr. 6, 2017

(30) Foreign Application Priority Data
Oct. 17, 2014 (EP) 14189300.8

(51) Int. Cl. G01R 33/422; G01R 33/3415; A61B 5/055; G01R 33/34; G01R 33/36; G01R 33/345

(52) U.S. Cl. CPC G01R 33/422; G01R 33/365; G01R 33/345; A61B 5/055; G01R 33/34076; G01R 33/3415

(57) ABSTRACT

The present invention provides a radio frequency (RF) coil (140) for applying an RF field to an examination space (116) of a magnetic resonance (MR) imaging system (110) and/or for receiving MR signals from the examination space (116), whereby the RF coil (140) is provided having a tubular body (142), the RF coil (140) is segmented in a longitudinal direction (154) of the tubular body (142) into two coil segments (146), and the two coil segments (146) are spaced apart from each other in the longitudinal direction (144) of the tubular body (142), whereby a gap (148) is formed between the two coil segments (146). The present invention further provides a magnetic resonance (MR) imaging system (110) comprising at least one radio frequency (RF) coil (140) as specified above. The present invention still further provides a medical system (200) comprising the above magnetic resonance (MR) imaging system (110) and a medical device (202), which is arranged to access to the examination space (116) of the magnetic resonance (MR) imaging system (110) through the gap (148) of the RF coil (140). Even further, the present invention provides a method for applying a radio frequency (RF) field to an examination space (116) of a magnetic resonance (MR) imaging system (110), comprising the steps of providing at least one above radio frequency antenna device (140), and commonly controlling the two RF coil segments (146) to provide a homogenous B₁ field within the examination space (116), in particular within the gap (148).

### FIELD OF THE INVENTION

The invention pertains to a radio frequency (RF) coil for use in an examination space of a magnetic resonance (MR) imaging system, an MR imaging system employing at least one such RF coil, a medical system employing such an MR imaging system and a medical device, and a method for applying a radio frequency field to an examination space of a magnetic resonance imaging system.

### BACKGROUND OF THE INVENTION

A state of the Art design of a magnetic resonance (MR) imaging system is for example an MR imaging system with a magnetic field strength of 3 Tesla. This state of the art MR imaging system employs e.g. a two-channel radio frequency (RF) body coil, which uses two geometrically decoupled feeding positions of a birdcage for RF-shimming. This technique provides a high field homogeneity and enables clinical imaging for additional applications at high field strengths. Although such MR imaging systems provide good imaging results, nowadays additional use cases for MR imaging systems emerge, which are the basis for additional requirements when designing an MR imaging system.

For example, the usage of MR imaging systems is becoming more and more common in the area of medical treatments, where the treatment is directed to a desired location of a subject of interest under guidance of an MR imaging system. E.g., in radiation therapy, an applicable dose can be directed to an exactly desired location, so that apart from the location, also the dose itself can be supervised during the treatment. Nevertheless, the applied radiation also affects the materials of the MR imaging system, so that for example increasing aging of material of the RF body coil may occur due to the applied radiation

Furthermore, also in diagnostic appliances, additional equipment can be required, which has to access the examination space. For example, bio sensors including e.g. a camera can be employed to supervise breathing or heartbeat of the subject of interest. These sensors preferably provide their sensor information from the subject of interest within the RF coil, where access to the subject of interest can be limited. Furthermore, connection of these sensor can require cabling, which may interfere with the fields generated by the MR imaging system, thereby reducing image quality of the MR imaging system.

### SUMMARY OF THE INVENTION

It is an object of the invention to provide an RF coil, an MR imaging system having such an RF coil, and a medical system including such an MR imaging system, which enable efficient treatment and/or diagnosis when using MR imaging systems, and which are less susceptible to altering e.g. through applied radiation in medical treatments.

This object is achieved by a radio frequency (RF) coil for applying an RF field to an examination space of a magnetic resonance (MR) imaging system and/or for receiving MR signals from the examination space, whereby the RF coil is provided having a tubular body, the RF coil is segmented in a longitudinal direction of the tubular body into two coil segments, and the two coil segments are spaced apart from each other in the longitudinal direction of the tubular body, whereby a gap is formed between the two coil segments. The RF coil could be a body coil, but could also be a local coil, e.g. a head coil. Preferably such head coil would comprise openings or a shape such that the shoulders of a patient can be fit in.

This object is also achieved by a magnetic resonance (MR) imaging system, comprising a tubular examination space provided to position a subject of interest therein, an RF screen for shielding the examination space, a magnetic gradient coil system for generating gradient magnetic fields superimposed to the static magnetic field, and a main magnet for generating a static magnetic field, whereby the RF screen, the magnetic gradient coil system and the main magnet are positioned in this order in a direction radially outward around the examination space, wherein the magnetic resonance (MR) imaging system comprises at least one radio frequency (RF) coil as specified above.

This object is further achieved by a medical system comprising a magnetic resonance (MR) imaging system as specified above, and a medical device, which is arranged to access the examination space of the magnetic resonance (MR) imaging system through the gap of the RF coil.

This object is also achieved by a method for applying a radio frequency (RF) field to an examination space of a magnetic resonance (MR) imaging system, comprising the steps of providing at least one radio frequency antenna device as specified above, and commonly controlling the two RF coil segments to provide a homogenous B₁ field within the examination space, in particular within the gap.

This object is still further achieved by a software package for upgrading a magnetic resonance (MR) imaging system, whereby the software package contains instructions for controlling the MR imaging system according to the above method.

Accordingly, with the gap provided between the two RF coil segments, the use of further devices used for e.g. medical treatment or analysis is facilitated, since treatments and/or analysis with medical devices can be performed through the gap. Hence, interferences with MR imaging system, in particular the RF coil, can be reduced. E.g. radiation applied to the examination space in conventional MR imaging systems employing conventional RF coils has to throughpass the material of the MR imaging systems and the conventional RF coil. Furthermore, when the radiation applied to the examination space in conventional MR imaging systems employing conventional RF coils throughpasses the conventional MR imaging system employing the conventional RF coil, the radiation alters the material of the MR imaging system and the conventional RF coil. Accordingly, accelerated aging of the materials occurs. These effects can be avoided, when the radiation is not directed towards the two RF coil segments, but passes through the gap between the two RF coil segments. Hence, the gap provides transparency for radiation therapy applications such as MR imaging guided linac or proton therapy. Furthermore, the localization of bio sensors such as camera detectors for the detection of motion (breathing, heart beat) can be facilitated by the gap. Another advantage of the proposed concept is a homogeneous attenuation of radiation. In case of using a state of the art RF coil, the attenuation is stronger in case of radiating through e.g. a coil conductor compared to radiating through air. This makes the treatment less efficient and less accurate. Having the RF coil separated into two segments with a gap in between, the radiation does not have to pass through e.g. a coil conductor, so that it is attenuated equally at different circumferential positions.

The RF coil segments are preferably provided having essentially the same length in the longitudinal direction of the tubular body. Hence, the gap preferably results in a central area of the RF coil, which facilitates the provisioning of a homogenous B1 field. Furthermore, each of the RF coil segments itself may be separated into individual segments. The RF coil segments can be provided simply as a separation of a state of the Art RF coil. Preferably, the RF coil segments are provided with individual feeding ports. The RF coil segments in principal refer to an electrical separation of the RF coil into two RF coil segments, so that resonators of the RF coil segments are spaced apart from each other by the gap. Hence, the RF coil segments can be provided as single components, where the two RF coil segments are mechanically interconnected. Nevertheless, the two RF coil segments can also be mechanically split into two individual components.

The RF coil segments typically comprise rungs extending in a longitudinal direction of the RF coil. The rungs are typically provided at the outer circumferential surface of the RF coil. A set of typically 8 or 16 rungs is equally spaced apart in a circumferential direction of the RF coil. In general, the number of rungs is a multiple of four. The rungs are preferably arranged in parallel to the longitudinal direction of the RF coil. In an alternative embodiment, the rungs are arranged with an angular displacement out of the longitudinal direction of the RF coil, resulting in a “diagonal” arrangement of the rungs. The angular displacement can be up to 20° out of the longitudinal direction of the RF coil. The rungs are provided typically with a distance of few centimeters, preferably two to four centimeters, from the RF screen. The RF screen can be integral part of the RF coil, or a component of the MR imaging system. The distance of the rungs to the RF screen can be held variable for optimization purposes.

There are different ways to build up a z-segmented RF coil, e.g. a bodycoil. The individual RF coil segments can be made of TEM-resonators and/or birdcage resonators. Hence, the two RF coil segments can be made of TEM-resonators, birdcage resonators, or a combination thereof. This does not change the general behavior of the RF coil having two segments in its longitudinal direction. The longitudinal direction is usually referred to as z-direction. Furthermore, each RF coil segment itself may be provided having multiple RF coil segments. Hence, the RF coil can be provided e.g. with four RF coil segments, whereby the gap is provided in a central region between the RF coil segments, e.g. with two RF coil segments on each side of the gap.

The coil segments do not need to be spaced evenly around the tubular body. For example by providing a lower number of coil segments on a first part of the RF coil compared to other parts of the RF coil, extra space for treatment delivery can be provided in the first part.

The RF coil allows efficient parallel image reconstruction techniques such as the SENSE algorithm in the longitudinal direction, i.e. in the z-direction, with a reduction factor of two. The SENSE algorithm is known in the art. Since each RF coil segment only covers a 50% of an examination space, an increase in the signal-to-noise ratio (SNR) is likely to occur, assuming that a patient loading is dominant. Nevertheless, also in cases where coil noise is dominant, an increase in the signal-to-noise ratio (SNR) is likely to occur. This might happen e.g. in case of using a very small distance to the RF-screen. The SNR is typically proportional to the sqrt(Q), i.e. the square root of the quality factor Q of the coil resonance. Typical quality factors Q are in the range of 300-600 in case of empty coils. Due to patient loading, the quality factor Q may be decreased by a factor of about 2 to 6. For higher reduction factors in a left/right (L-R) and anterior/posterior (A-P) direction, the coils have to be configured in a degenerate design. Also RF shimming is feasible depending on the number of available independent RF channels of the RF coil. For an RF coil having four independent RF channels, RF shimming can be achieved e.g. along the z-direction of the RF coil, i.e. the longitudinal direction of the RF coil, and the x-y direction of the RF coil.

In the MR imaging system, the RF screen, the magnetic gradient coil system, and the main magnet, are typically arranged concentrically to surround the examination space. Overall, a typical full setup of the MR imaging system comprises the subject of interest, when located in the examination space, a full body RF coil used as receive and transmit coil, e.g. a full body coil, the RF screen, the magnetic gradient coil system, and the main magnet, when starting at a center of the examination space and moving in a radial direction. In an alternative embodiment, the MR imaging system comprises additionally a local RF coil, which is typically used as receive coil only, and which is located within the RF coil provided as full body coil to surround the subject of interest at least partially. In this alternative embodiment, the RF coil provided as full body coil and is used as transmit coil only. Furthermore, the gradient coil system may be provided with shim coils, which are provided at an radially outer area of the gradient coil system.

In the medical system, the medical device can be any suitable kind of device, e.g. a diagnostic/analytic or therapeutic device. The diagnostic devices may comprise any suitable kind of diagnostic/analytic devices including devices for detection of breathing/breath-hold, heartbeat detection devices, positron emission tomography (PET) devices, in particular PET receivers, bio sensors, camera detectors, or others. The therapeutic devices may comprise any suitable kind of therapeutic devices including radiotherapy systems, linear accelerator (LINAC) devices, proton treatment devices, MR hyperthermia devices or others. In an alternative embodiment, the gap can also be used for positioning RF amplifiers of the MR imaging system.

The medical device can be located depending on size, form and particular needs for accessing the examination space and/or a subject of interest located in the examination space. Accordingly, the medical device can be located in the gap, or the medical device can access the examination space and/or a subject of interest through the gap. For example, a typical LINAC device is provided rotatable around the examination space and the accelerated particles can be directed to the subject of interest through the gap without the risk of interfering with components of the RF coil.

In other cases, the medical device can be positioned e.g. within the examination space, like an MR hyperthermia device. The MR hyperthermia device can be accessed and/or connected through the gap, thereby reducing coupling with the MR imaging device, in particular with the RF coil. Since individual coil elements of the two segments of the RF coil are not directly under the applicator, i.e. the MR hyperthermia device in this case, a good decoupling can be achieved.

According to a preferred embodiment, the two coil segments are arranged relative to each other with a rotational angle around the longitudinal axis of the tubular body. Accordingly, rungs of the two RF coil segments, which extend in the longitudinal direction of the RF coil, can be aligned between the two RF coil segments, or they can be arranged such that rungs from the one RF coil segment point in a direction between the rungs of the other RF coil segment.

According to a preferred embodiment, the two coil segments are coupled together to generate a conventional birdcage field. Preferably, the two coil segments are coupled by a (n times) lambda/2 transmission line, which provides one possibility to couple the two RF coil segments to generate a conventional birdcage field. With the lambda/2 coupling, the two coil segments can be driven like a conventional coil without the gap, e.g. like a conventional birdcage coil. Hence, the RF coil can be used to substitute conventional RF coils in existing MR imaging systems. The replacement can be performed even though the MR imaging system is a stand-alone device, which is not used as part of a medical system, i.e. even though the MR imaging system is not used together with an additional therapeutic or diagnostic device, which requires access to the examination space.

According to a preferred embodiment, the two coil segments are decoupled from each other and driven independently. Decoupling of the two RF coil segments enables that the RF coil as a whole can be driven as a four channel coil array. Hence, excitation of RF fields can be realized in a very accurate and efficient way.

According to a preferred embodiment, the two coil segments can be driven with separate RF power amplifiers or using a hardware combiner or a splitter. Hence, the two coil segments can be driven independently with the two RF power amplifiers. Alternatively, the two coil segments are driven in a combined way with just a single driver.

According to a preferred embodiment, the RF coil is provided as a hybrid RF coil, having a hybrid design of a birdcage coil and a TEM coil, whereby the RF coil is TEM-like in its center region and birdcage-like at its end regions in the longitudinal direction. Accordingly, the two RF coil segments are provided with a conductive ring in the area located apart from the gap, and conductive rungs extend from the conductive ring in the direction of the gap. The conductive rungs are coupled to the RF shield, which can be part of the RF coil itself, or which can be part of the MR imaging system. The RF coil comprises an RF screen, to which the conductive rungs are coupled at their ends facing the gap. Alternatively, the screen can be part of the MR imaging system, and the conductive rungs are coupled at their end facing the to the RF screen. Hence, for the overall RF coil results a hybrid design, which is TEM-like in its center region and birdcage-like at the ends in the longitudinal direction. Typical QBC-dimensions of a conventional RF coil comprise a shield radius of 370 mm, a coil radius of 355 mm, and a coil length of 500 mm. For such a typical, conventional RF coil, a gap of approximately 20 cm can be achieved without affecting the operation and the imaging quality of the MR imaging system. Preferably, the gap has a width in the longitudinal direction of the RF coil of at least 5 cm, further preferred gap has a width of at least 10 cm, and still further preferred the gap has a width of 15 cm to 20 cm. The above coil dimensions are given by way of example only. For other coil dimensions, the width of the gap may be different.

According to a preferred embodiment, at least one segment of the RF coil is provided as a multi-element transmit-array. Hence, in combination with a hardware combiner, a decoupling of the two RF coil segments is presumably obsolete, since the coupling between the individual RF coil segments is low.

According to a preferred embodiment at least one of the RF screen, the magnetic gradient coil system and the main magnet are segmented in the longitudinal direction of the examination space into two segments, which are spaced apart from each other in the longitudinal direction of the tubular body, whereby a gap is formed between the two segments. Preferably, the gap provided between the RF screen, the magnetic gradient coil system and/or the main magnet are aligned with the gap between the two RF coil segments. Accordingly, the advantages achieved by the gap between the separation of the RF coil into two RF coil segments apply also to the RF screen, the magnetic gradient coil system, or the main magnet. In the case of the RF screen, RF screen segments can be provided as single components, where the two RF screen segments are mechanically interconnected. Nevertheless, the two RF screen segments can also be mechanically split into two individual components. The longitudinal direction of the examination space and of the tubular body are aligned, i.e. the directions are identical.

According to a preferred embodiment, the RF screen is segmented in the longitudinal direction of the examination space into two RF screen segments. The two RF screen segments are spaced apart from each other in the longitudinal direction of the tubular body, whereby a gap is formed between the two RF screen segments, and an alternative RF screen element is provided to connect the two RF screen segments through the gap. To achieve an efficient RF screening, the RF screen is typically provided as a metal sheet or a metal web with a tight web structure, which is not transparent to RF fields. Furthermore, as already discussed above in respect to the rungs, also the RF screen is not transparent e.g. in respect to radiation when using a LINAC or other radiation devices together with the MR imaging system. To increase the transparency of the RF screen for radiation, the alternative RF screen element can be provided made from a non-conductive material, a mesh-like screen made of conductive material can be used, or a conductive layer with a higher transparency can be used. For example, a thin conductive layer made of copper with a thickness of about 15-40 μm, when used as alternative RF screen element, is almost transparent for radiation from a LINAC device. In an alternative embodiment, the alternative RF screen element can be provided as an overlap area of parts of the two RF screen segments, which overlap through the gap. In a further alternative embodiment, at least one conductive strip can be provided to galvanically connect the two RF screen segments through the gap. Preferably, multiple conductive strips are provided, which are spaced apart in a circumferential direction of the RF screen. Accordingly, an alternative RF screen element is formed as an element having at least one window in the gap. Furthermore, a capacitive coupling can be provided between the two RF screen segments. Hence, an electrical connection between the RF screen segments can be omitted, which enables the use of different kinds of alternative RF screen elements. The longitudinal direction of the examination space and of the tubular body are aligned, i.e. the directions are identical.

According to a preferred embodiment, the RF screen, the magnetic gradient coil system, and the main magnet are segmented in the longitudinal direction of the examination space into two segments each, the two segments are spaced apart from each other in the longitudinal direction of the tubular body, whereby a gap is formed between each of the two segments, and the two RF screen segments extend along the gap in a ring-like manner in a direction radially outward of the examination space. This design of the RF screen, i.e. of the two RF screen segments provides an extended RF screening in the direction of the gap to provide a shielding to the gradient coil. The slot formed in the gap is narrow compared to the typical dimensions of the RF coil and provides a suppression of radiation. Preferably, the RF screen segments or folded radially outwards. Preferably, the rungs of the RF coil segments are connected to the RF screen, so that an RF current can flow back via the RF screen, so that the gap can also be provided in the RF screen. The longitudinal direction of the examination space and of the tubular body are aligned, i.e. the directions are identical.

### BRIEF DESCRIPTION OF THE DRAWINGS

These and other aspects of the invention will be apparent from and elucidated with reference to the embodiments described hereinafter. Such an embodiment does not necessarily represent the full scope of the invention, however, and reference is made therefore to the claims and herein for interpreting the scope of the invention.

In the drawings:

FIG. 1 is a schematic illustration of a part of a generic embodiment of a magnetic resonance (MR) imaging system,

FIG. 2 is a schematic illustration of an RF coil according to a first embodiment,

FIG. 3 is a perspective view of an RF coil together with an RF screen according to a second embodiment,

FIG. 4 is a perspective view of the RF coil of FIG. 3 showing a simulated current distribution at a given point in time,

FIG. 5 is a perspective view of the RF coil of FIG. 3 showing a simulated current distribution at a given point of time for an RF coil with coupled and decoupled RF coil segments on the left and right side, respectively,

FIG. 6 is a diagrammatic illustration of scattering parameters in the top diagrams and smith charts in the bottom diagrams for the RF coil with coupled and decoupled RF coil segments on the left and right side, respectively,

FIG. 7 is a schematic illustration of an RF coil according to a third embodiment employed as multi-element transmit-array with capacitive decoupling,

FIG. 8 is a schematic illustration of an RF coil according to a fourth embodiment employed as multi-element transmit-array with inductive decoupling,

FIG. 9 is a perspective view of an RF coil together with an RF screen according to a fifth embodiment,

FIG. 10 is a diagrammatic illustration of simulated B1 fields using the RF coil of the fifth embodiment,

FIG. 11 is a diagrammatic illustration of input impedance over the frequency using the RF coil of the fifth embodiment,

FIG. 12 is a schematic illustration of a medical system comprising an MR imaging system with an RF coil and a medical device according to a sixth embodiment,

FIG. 13 is a schematic illustration of an MR imaging system with an RF coil and segmented RF screen with an alternative RF screen element located therebetween according to a seventh embodiment,

FIG. 14 is a schematic illustration of an RF coil with two RF coil segments and a decoupling circuit according to an eighths embodiment, and

FIG. 15 is a schematic illustration of an RF screen with two RF screen segments together with an alternative RF screen element according to a ninth embodiment.

### DETAILED DESCRIPTION OF EMBODIMENTS

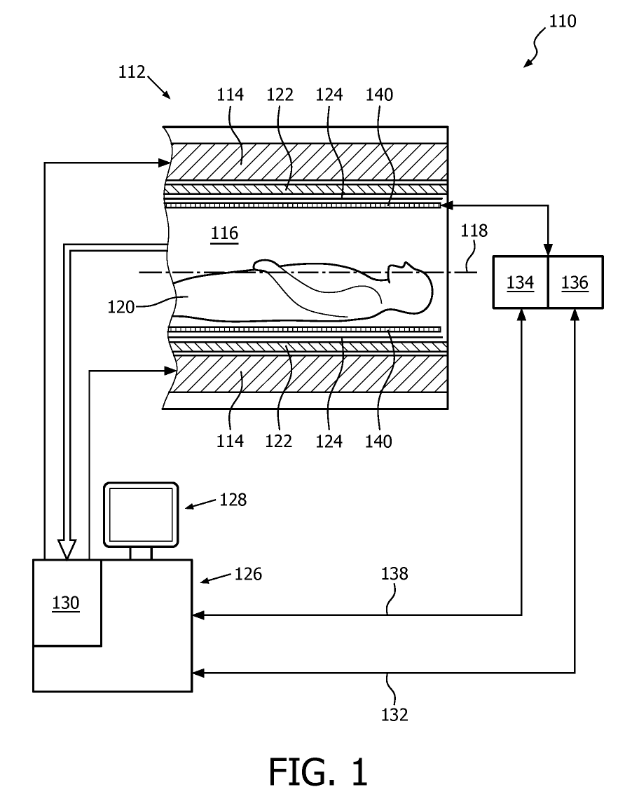

FIG. 1 shows a schematic illustration of a part of an embodiment of a magnetic resonance (MR) imaging system 110 comprising an MR scanner 112. The MR imaging system 110 is described here generically as a basis for all further embodiments.

The MR imaging system 110 includes a main magnet 114 provided for generating a static magnetic field. The main magnet 114 has a central bore that provides an examination space 116 around a center axis 118 for a subject of interest 120, usually a patient, to be positioned within. In this embodiment, the central bore and therefore the static magnetic field of the main magnet 114 have a horizontal orientation in accordance with the center axis 118. In an alternative embodiment, the orientation of the main magnet 114 can be different, e.g. to provide the static magnetic field with a vertical orientation. Further, the MR imaging system 110 comprises a magnetic gradient coil system 122 provided for generating gradient magnetic fields superimposed to the static magnetic field. The magnetic gradient coil system 122 is concentrically arranged within the bore of the main magnet 114, as known in the art.

Further, the MR imaging system 110 includes a radio frequency (RF) coil 140 designed as a whole-body coil having a tubular body. In an alternative embodiment, the RF coil 140 is designed as a head coil or any other suitable coil type for use in MR imaging systems 110. The RF coil 140 is provided for applying an RF magnetic field to the examination space 116 during RF transmit phases to excite nuclei of the subject of interest 120, which shall be covered by MR images. The RF coil 140 is also provided to receive MR signals from the excited nuclei during RF receive phases. In a state of operation of the MR imaging system 110, RF transmit phases and RF receive phases are taking place in a consecutive manner. The RF coil 140 is arranged concentrically within the bore of the main magnet 114. As is known in the art, a cylindrical metal RF screen 124 is arranged concentrically between the magnetic gradient coil system 122 and the RF coil 140.

In this context, it is to be noted that the RF coil 140 has been described as transmit and receive coil. Nevertheless, the RF coil 140 can also be provided as transmit or receive coil only.

Moreover, the MR imaging system 110 comprises an MR image reconstruction unit 130 provided for reconstructing MR images from the acquired MR signals and an MR imaging system control unit 126 with a monitor unit 128 provided to control functions of the MR scanner 112, as is commonly known in the art. Control lines 132 are installed between the MR imaging system control unit 126 and an RF transmitter unit 134 that is provided to feed RF power of an MR radio frequency to the RF antenna device 140 via an RF switching unit 136 during the RF transmit phases. The RF switching unit 136 in turn is also controlled by the MR imaging system control unit 126, and another control line 138 is installed between the MR imaging system control unit 126 and the RF switching unit 136 to serve that purpose. During RF receive phase, the RF switching unit 136 directs the MR signals from the RF coil 140 to the MR image reconstruction unit 130 after pre-amplification.

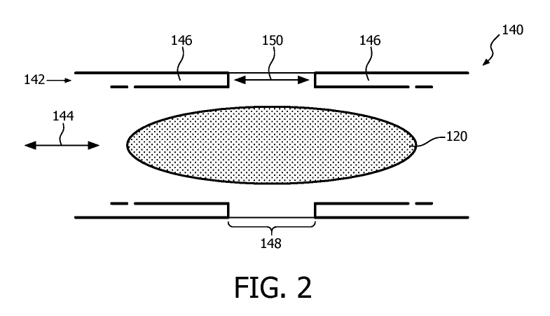

FIG. 2 shows an RF coil 140 for applying an RF field to the examination space 116 of the MR imaging system 110 and for receiving MR signals from the examination space 116 according to a first embodiment. The subject of interest 120 is located within the RF coil 140. The RF coil is provided having a tubular body 142 and is segmented in a longitudinal direction 144 of the tubular body 142 into two RF coil segments 146. The longitudinal direction 144 is usually referred to as z-direction. The two RF coil segments 146 are spaced apart from each other in the longitudinal direction 144 of the tubular body 142, whereby a gap 148 is formed between the two RF coil segments 146. Accordingly, the two RF coil segments 146 are spaced apart a distance 150, as shown in FIG. 2.

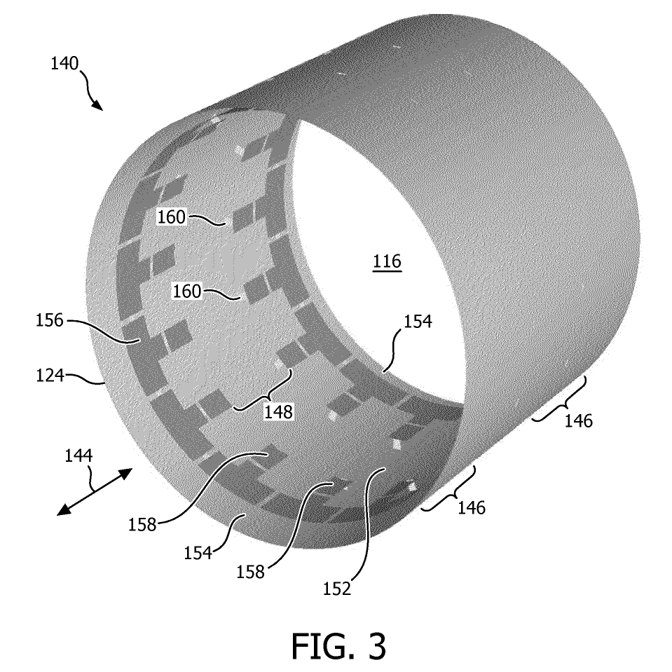

FIG. 3 shows an RF coil 140 for applying an RF field to the examination space 116 of the MR imaging system 110 and for receiving MR signals from the examination space 116 according to a second embodiment. Principles of the RF coil 140 according to the first embodiment also apply to the RF coil 140 of the second embodiment, unless otherwise stated.

The RF coil of the second embodiment is provided as a hybrid RF coil 140 having a hybrid design of a birdcage coil and a TEM coil. As can be seen in FIG. 3, the RF coil 140 is TEM-like in its center region 152 and birdcage-like at its end regions 154 in the longitudinal direction 144. Accordingly, the two RF coil segments 146 are provided with a conductive ring 156 in the end regions 154, which are located apart from the gap 148, and conductive rungs 158 extending from the conductive ring 156 in the direction of the gap 148. Each RF coil segment 146 in this embodiment is provided with a set of 16 conductive rungs 158, which are equally spaced apart in a circumferential direction of the RF coil 140. In an alternative embodiment, the RF coil 140 is provided with two sets of eight conductive rungs 158, i.e. one set of eight conductive rungs 158 is provided in each RF coil segment 146. The conductive rungs 158 are provided with a distance of few centimeters, preferably two to four centimeters, from the RF screen 124.

The conductive rungs 158 are coupled to the RF screen 124 at their end facing the gap 148 with coupling capacitors 160. In an alternative embodiment, the conductive rungs 158 are galvanically connected or capacitively coupled to the RF screen 124, e.g. using pads close to the RF screen 124. In a further alternative embodiment, the RF screen 124 is part of the RF coil 140 itself. Hence, for the RF coil 140 results a hybrid design, which is TEM-like in its center region 152 and birdcage-like at the end regions 154. The RF coil 140 is provided with the RF screen 124 having radius of 370 mm, the RF coil 140 having radius of 355 mm and a coil length of 500 mm. The gap 148 has a length of approximately 20 cm. Accordingly, each RF coil segment 146 has a coil segment length of approximately 15 cm, e.g. RF coil length of 50 cm minus the length of the gap of 20 cm divided by 2.

As can be seen in detail in FIG. 3, the RF coil segments 146 are provided having essentially the same length in the longitudinal direction 144 of the tubular body 142. The RF coil segments 146 are provided with individual feeding ports, which are not shown in this figure. The RF coil segments 146 refer to an electrical separation of the RF coil 140 into two RF coil segments 146, so that resonators of the RF coil segments 146 are spaced apart from each other by the gap 148. The RF coil segments 146 in this embodiment are also mechanically split into two individual RF coil segments 146. In an alternative embodiment, the RF coil elements 146 are provided as single components, where the two RF coil segments 146 are mechanically interconnected.

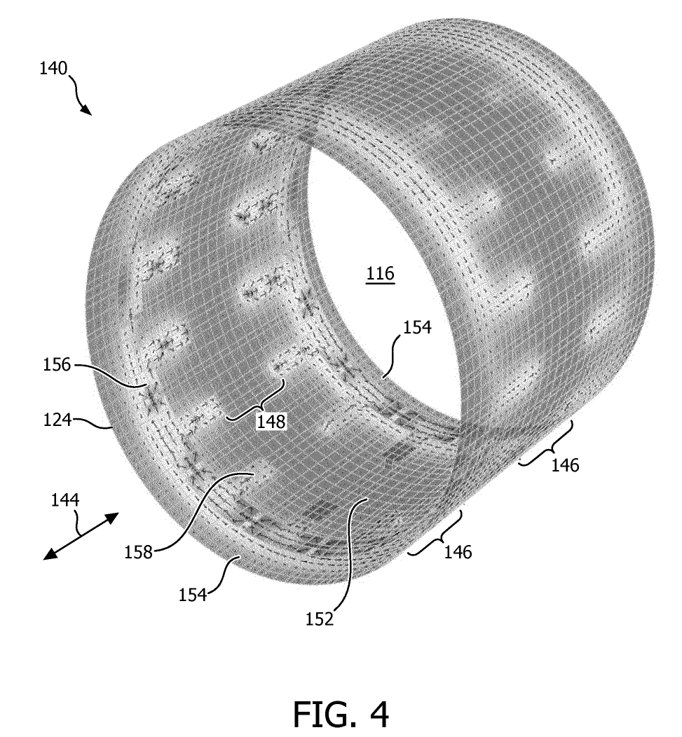

FIG. 4 shows a simulated current distribution at a given point in time for the RF coil 140 of the second embodiment. As can be seen in FIG. 4, the currents through the two RF coil segments 146 are almost identical.

General techniques for decoupling of the RF coil segments 146 are known e.g. from US 2013/0063147 A1, which is incorporated herein by reference.

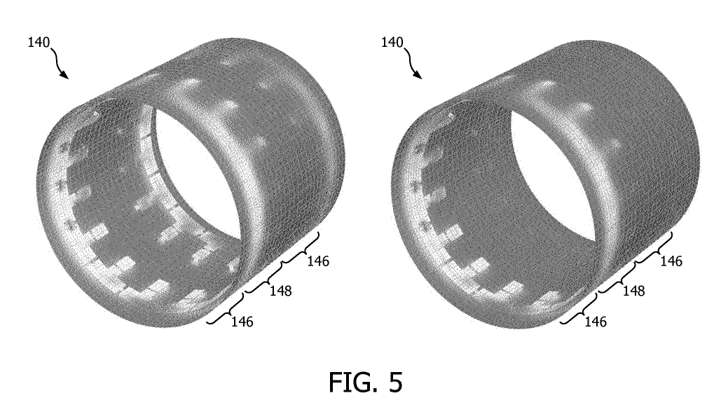

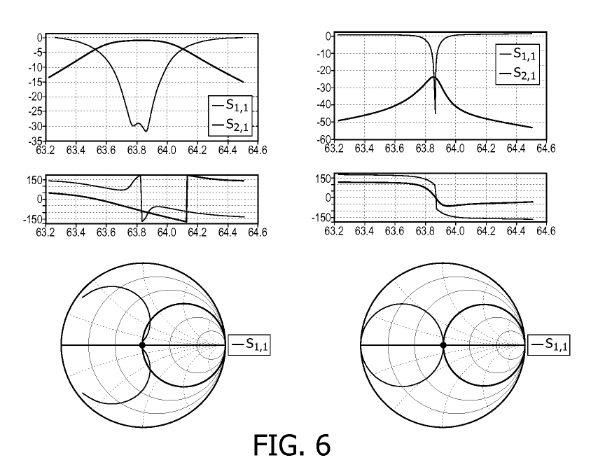

In FIG. 5 a simulated current distribution at a given point in time for the RF coil 140 of the second embodiment is shown. FIG. 6 shows the current distribution for the RF coil 140 with coupled and decoupled RF coil segments 146 on the left and right side, respectively.

In FIG. 6 an illustration of scattering parameters is given in the top diagrams for the RF coil 140 with coupled and decoupled RF coil segments 146 on the left and right side, respectively, in accordance with the drawing of FIG. 5.

Furthermore, in FIG. 6 an illustration of smith charts is given in the bottom diagrams for the RF coil 140 with coupled and decoupled RF coil segments 146 on the left and right side, respectively, in accordance with the drawing of FIG. 5.

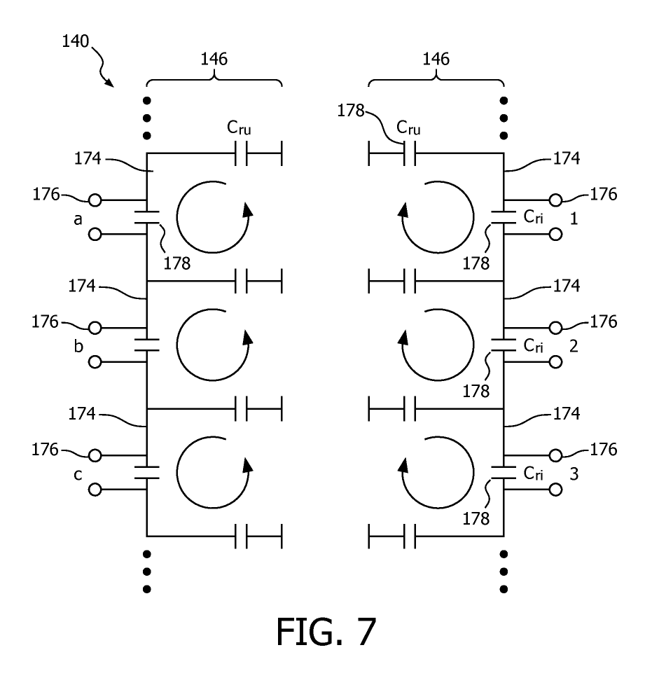

FIG. 7 shows an RF coil 140 for applying an RF field to the examination space 116 of the MR imaging system 110 and for receiving MR signals from the examination space 116 according to a third embodiment. Principles of the RF coil 140 according to the first and second embodiments also apply to the RF coil 140 of the third embodiment, unless otherwise stated.

The RF coil 140 according to the third embodiment is employed as multi-element transmit-array with capacitive decoupling. Hence, multiple elements are provided as meshes 174, which can be fed via feeding ports 176. Coupling capacitors 178 are provided in the meshes 174, which are also denoted Cᵣᵢ and Cᵣᵤ, to easily distinguish the coupling capacitors 178. The RF coil 140 can be provided as degenerate RF coil 140 by choosing the correct ratio Cᵣᵢ/Cᵣᵤ, so that the individual meshes 174 are decoupled. Accordingly, each individual mesh 174 in the two RF coil segments 146 can be driven independently by a parallel Tx/Rx RF system.

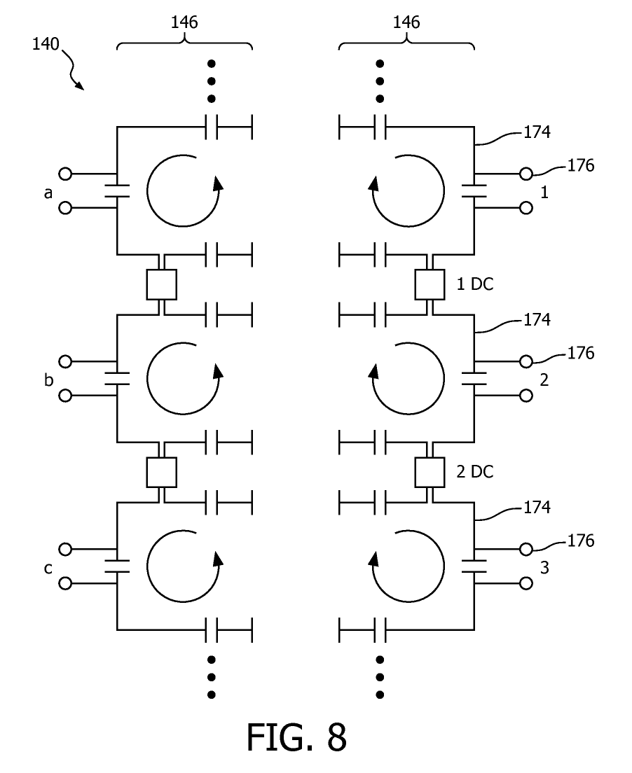

FIG. 8 shows an RF coil 140 for applying an RF field to the examination space 116 of the MR imaging system 110 and for receiving MR signals from the examination space 116 according to a fourth embodiment. Principles of the RF coil 140 according to the third embodiment also apply to the RF coil 140 of the fourth embodiment, unless otherwise stated.

The RF coil 140 of the fourth embodiment differs from the RF coil 140 of the third embodiment in the decoupling. According to FIG. 8, inductive decoupling transformers 180 are provided between adjacent meshes 174. Apart from this difference, the RF coils 140 of the third and fourth embodiment are identical.

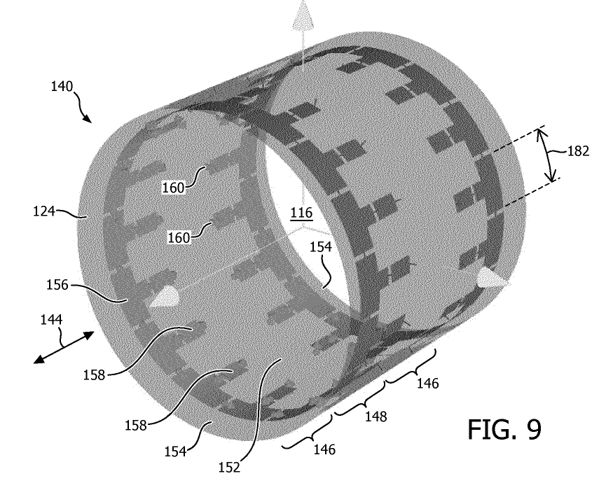

FIG. 9 shows an RF coil 140 for applying an RF field to the examination space 116 of the MR imaging system 110 and for receiving MR signals from the examination space 116 according to a fifth embodiment. Principles of the RF coil 140 according to the above described embodiments also apply to the RF coil 140 of the fifth embodiment, unless otherwise stated.

The RF coil 140 of the fifth embodiment is almost identical to the RF coil 140 of the second embodiment. The RF coils 140 of the fifth and second embodiments differ in that the two coil segments 146 of the fifth embodiment are arranged relative to each other with a rotational angle 182 around the longitudinal axis of the tubular body 142. Accordingly, the conductive rungs 158 from the one RF coil segment 146 point in a direction between the conductive rungs 158 of the other RF coil segment 146.

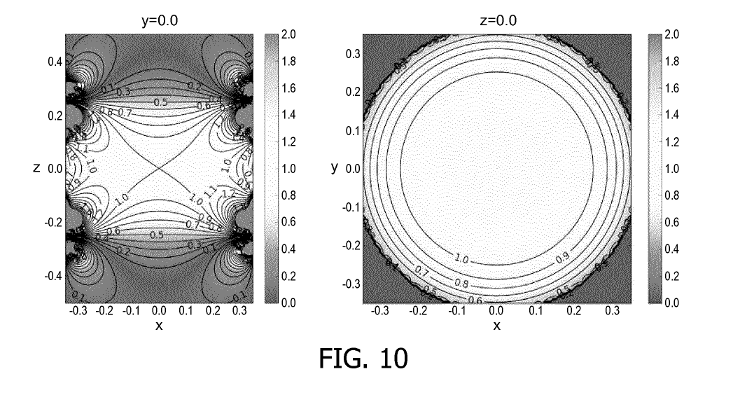

In FIG. 10 can be seen a diagrammatic illustration of simulated B1 fields using the RF coil of the fifth embodiment. Coronal and transversal B1 field homogeneity of simulated coil design is shown in the right and left diagram of FIG. 10, respectively. Contour lines are plotted in 10% steps compared to isocenter field. As can be seen, in the provided gap 148 of the RF coil 140, a homogeneous radial field is provided. On the central (z) axis, the field is very similar compared to a standard birdcage coil. Accordingly, the two RF coil segments 146 are commonly controlled to provide a homogenous B₁ field within the examination space 116, in particular within the gap 148.

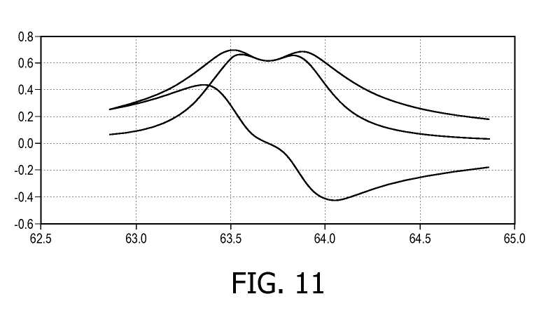

In FIG. 11 can be seen input impedance over the frequency using the RF coil 140 of the fifth embodiment. The Input impedance shows two very close resonances. The homogeneous mode is tuned to 63.86 MHz, the second mode appears at 63.53 MHz. Accordingly, mode separation is generated by separating the RF coil 140 into two RF coil segments 146. The two modes are split by just approximately 300 kHz. Hence, for the RF coil 140 of the fifth embodiment, four-port feeding for a quadrature coil is proposed. Alternatively, additional decoupling can be performed for using the coil like a 2×2=4 channel z-segmented bodycoil.

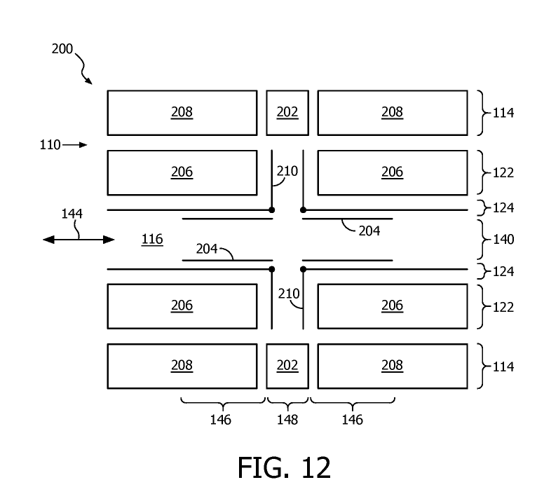

FIG. 12 schematically shows a medical system 200 according to a sixth embodiment. The medical system 200 comprises the above MR imaging system 110 with the RF coil 140 and a medical device 202.

As can be seen in FIG. 12, the MR imaging system 110 comprises an RF coil 140 as described above in respect to the first to fifth embodiment, RF screen 124, a magnetic gradient coil system 122 and a main magnet 114, as described above in respect to FIG. 1. The RF coil 140, the RF screen 124, the magnetic gradient coil system 122, and the main magnet 114 are arranged concentrically to surround the examination space 116. The RF coil 140, the RF screen 124, the magnetic gradient coil system 122, and the main magnet 114 are segmented in the longitudinal direction 144 of the examination space 116 into two segments each, i.e. two RF coil segments 146, two RF screen segments 204, two gradient coil segments 206, and two magnet segments 208, which are all spaced apart from each other in the longitudinal direction 144 of the tubular body 142, so that a gap 148 is formed between the respective segments 146, 204, 206, 208. The gap 148 is provided as single gap 148 for the RF coil segments 146, the RF screen segments 204, the gradient coil segments 206 and the main magnet segments 208 by aligning the respective segments 146, 204, 206, 208.

As can be further seen in FIG. 12, the two RF screen segments 204 are provided each with a ring-like extension 210. The ring-like extensions 210 extend from the respective RF screen segments along the gap 148 in a direction radially outward of the examination space 116.

The medical device 202 is arranged to access the examination space 16 of the MR imaging system 110 through the gap 148 of the RF coil 140, the RF screen 124, the gradient coil system 122, and the main magnet 116. Accordingly, with the provided gap 148, application of the medical device to the subject of interest 116 can be performed through the gap 148, e.g. when using a medical treatment/therapeutic device as medical device 202 to apply medical treatment through the gap 148.

The medical device 202 can be any suitable kind of device, e.g. a diagnostic or therapeutic device. The therapeutic devices may comprise radiotherapy systems, LINAC devices, proton treatment devices, MR hyperthermia devices or others.

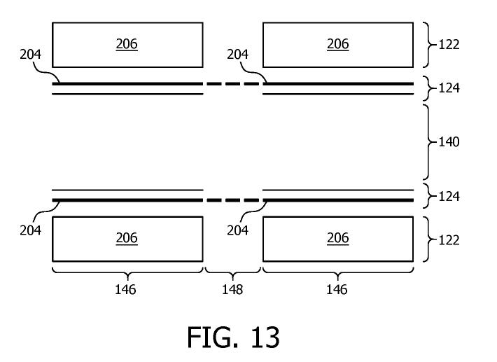

FIG. 13 schematically shows a medical system 200 according to a seventh embodiment. The medical system 200 comprises the above MR imaging system 110 with the RF coil 140 and a medical device 202 and only differs from the medical system 200 of the first embodiment in the design of the RF screen 124, as detailed out below.

As can be seen in FIG. 13, the RF screen 124 is separated into two RF screen segments 204, as described above in respect to the sixth embodiment and spaced apart from each other. The two RF screen segments 204 according to the seventh embodiment are interconnected with an alternative RF screen element 212 located therebetween. Hence, the alternative RF screen element 212 is provided to connect the two RF screen segments 204 through the gap 148. To increase the transparency of the RF screen 124 for radiation, the alternative RF screen element 212 can be provided made from a non-conductive material, a mesh-like RF screen element 212 made of conductive material can be used, or a conductive layer with a higher transparency can be used as alternative RF screen element 212.

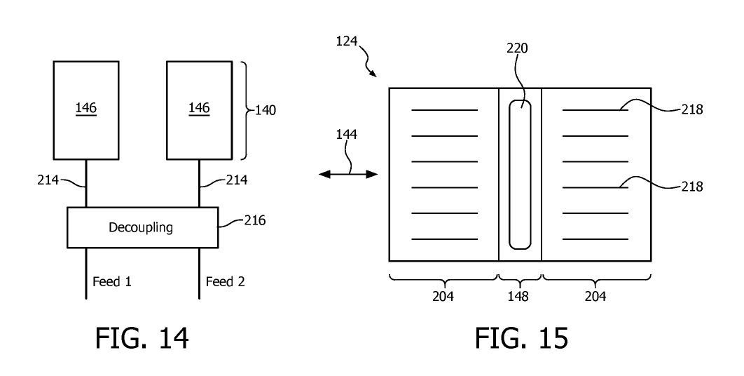

FIG. 14 schematically shows an RF coil 140 according to an eighth embodiment. The RF coil 140 is provided in accordance with the RF coils 140 of the above embodiments.

As can be seen in FIG. 14, the two RF coil segments 146 of the RF coil are decoupled using low loss cables 214, which are connected to a decoupling circuit 216. This prevents any cable or stripline connections between the two RF coil segments 146 via the gap 148. Each RF coil segment 146 is driven in quadrature mode or by separate independent transmitters. In an alternative embodiment, the RF coils segments 146 are decoupled via the gap 148 using thin stripline conductors or flexible thin PCB material, which provides low radiation and attenuation.

FIG. 15 shows an RF screen 124 of an RF coil 140 according to a ninth embodiment. The RF coil 140 and the RF screen 124 are provided in accordance with the above described embodiments. As can be seen in FIG. 15, the RF screen 124 is provided with two RF screen segments 204, which are spaced apart, thereby providing gap 148 therebetween. Each RF screen segment 204 is provided with structure extending in the longitudinal direction 144 to reduce gradient eddy currents and to allow RF current flow for mirror RF currents of the RF coil segments 146. In the gap, an opening 220 is provided for transparency of radiation. The opening 220 in this embodiment is provided without material in a window style. In alternative embodiments, the opening 220 is provided with a non-conductive material or conductive material like a thin mesh, or a thin conductive layer different from the RF screen segments 204.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCE SYMBOL LIST

110 magnetic resonance (MR) imaging system

112 magnetic resonance (MR) scanner

114 main magnet

116 RF examination space

118 center axis

120 subject of interest

122 magnetic gradient coil system

124 RF screen

126 MR imaging system control unit

128 monitor unit

130 MR image reconstruction unit

132 control line

134 RF transmitter unit

136 RF switching unit

138 control line

140 radio frequency (RF) coil

142 tubular body

144 longitudinal direction

146 RF coil segment

148 gap

150 distance

152 center region

154 end region

156 conductive ring

158 conductive rung

160 coupling capacitor

174 mesh

176 feeding port

178 coupling capacitor

180 inductive decoupling transformers

182 rotational angle

200 medical system

202 medical device

204 RF screen segment

206 gradient coil segment

208 magnet segment

210 ring-like extension

212 alternative screen element

214 low loss cable

216 decoupling circuit

218 structure

220 opening

1. A radio frequency (RF) coil for applying an RF field to an examination space of a magnetic resonance (MR) imaging system and/or for receiving MR signals from the examination space, whereby
the RF coil is provided having a tubular body,
the RF coil is segmented in a longitudinal direction of the tubular body (142) into a first and a second coil segment, spaced apart from each other in the longitudinal direction of the tubular body
whereby a gap is formed between the first and second coil segment, wherein the RF coil is provided as a hybrid RF coil having a hybrid design of a birdcage coil and a TEM coil, whereby
the RF coil is TEM-like in its center region and birdcage-like at its end regions in the longitudinal direction by providing the first and second coil segment with a first and second conductive ring respectively in an area located apart from the gap and by providing the first and second coil segment with first and second conductive rungs extending from the first and second conductive ring respectively in a direction of the gap, wherein the first and second conductive rungs are configured to be coupled to an RF screen at their ends facing the gap.

2. The radio frequency (RF) coil according to preceding claim 1, wherein the first and second coil segment are arranged relative to each other with an rotational angle around the longitudinal axis of the tubular body.

3. The radio frequency (RF) coil according to claim 1, wherein the first and second coil segment are coupled together to generate a conventional birdcage field.

4. The radio frequency (RF) coil according to claim 1, wherein the first and second coil segment are decoupled from each other and driven independently.

5. The radio frequency (RF) coil according to claim 1, wherein the first and second coil segment can be driven with separate RF power amplifiers or using a hardware combiner or a splitter.

6. The radio frequency (RF) coil according to claim 1, wherein at least one segment of the RF coil is provided as a multi-element transmit-array.

7. A magnetic resonance (MR) imaging system, comprising:
a tubular examination space provided to position a subject of interest therein,
an RF screen for shielding the examination space,
a magnetic gradient coil system for generating gradient magnetic fields superimposed to the static magnetic field, and
a main magnet for generating a static magnetic field,
whereby the RF screen, the magnetic gradient coil system and the main magnet are positioned in this order in a direction radially outward around the examination space, wherein
the magnetic resonance (MR) imaging system comprises at least one radio frequency (RF) coil according to claim 1.

8. The magnetic resonance (MR) imaging system according to preceding claim 7, wherein at least one of the RF screen, the magnetic gradient coil system and the main magnet are segmented in the longitudinal direction of the examination space into two segments, which are spaced apart from each other in the longitudinal direction of the tubular body, whereby a gap is formed between the two segments.

9. The magnetic resonance (MR) imaging system according to claim 7, wherein
the RF screen is segmented in the longitudinal direction of the examination space into two RF screen segments,
the two RF screen segments are spaced apart from each other in the longitudinal direction of the tubular body, whereby a gap is formed between the two RF screen segments, and
an alternative RF screen element is provided to connect the two RF screen segments through the gap.

10. The magnetic resonance (MR) imaging system according to claim 7, wherein
the RF screen, the magnetic gradient coil system and the main magnet are segmented in the longitudinal direction of the examination space into two segments each,
the two segments are spaced apart from each other in the longitudinal direction of the tubular body, whereby a gap is formed between each of the two segments, and
the two RF screen segments extend along the gap in a ring-like manner in a direction radially outward of the examination space.

11. A medical system comprising:
a magnetic resonance (MR) imaging system according to claim 7, and
a medical device, which is arranged to access to the examination space of the magnetic resonance (MR) imaging system through the gap of the RF coils.

12. A method for applying a radio frequency (RF) field to an examination space of a magnetic resonance (MR) imaging system, comprising the steps of
providing at least one radio frequency antenna device as claimed in claim 1, and
commonly controlling the two RF coil segments to provide a homogenous B1 field within the examination space, in particular within the gap.

13. A software package for upgrading a magnetic resonance (MR) imaging system, whereby the software package contains instructions for controlling the MR imaging system according to method claim 12.
